(19) United States
(12) Patent Application Publication
(10) Pub. No.: US 2021/0123882 A1
(43) Pub. Date: Apr. 29, 2021

(54) METHOD FOR PREPARING BIOSENSING MEMBRANE, BIOSENSING MEMBRANE AND MONITORING DEVICE

(71) Applicant: Sinocare Inc., Changsha, Hunan (CN)

(72) Inventor: ZhiQiang GAO, Changsha (CN)

(21) Appl. No.: 17/044,016

(22) PCT Filed: Apr. 25, 2019
(86) PCT No.: PCT/CN2019/084254

§ 371 Date: Sep. 30, 2020

(30) Foreign Application Priority Data
Jul. 27, 2018 (CN) 201810846227.6

(51) Int. Cl. G01N 27/327; G01N 27/31; C12N 9/04; C12N 9/02

(52) U.S. Cl. CPC G01N 27/3277; C12N 9/0069; C12N 9/0006; G01N 27/31

(57) ABSTRACT

A method for preparing a biosensing membrane: electrochemically activating and modifying an oxidoreductase, then performing a cross-linking treatment using a chemical cross-linking agent, and then coating on a surface of an electrode, thereby forming a biosensing membrane, wherein the chemical cross-linking agent is glutaraldehyde or polyethylene glycol diglycidyl ether. Also disclosed are a prepared biosensing membrane and monitoring device. The provided preparation method, or the biosensing membrane and monitoring device prepared by the preparation method are stable and durable, and may carry out a plurality of detections, and the foregoing biosensing membrane is particularly suitable to act as a biosensing membrane of a living body monitoring device.

### TECHNICAL FIELD

This disclosure generally relates to the technical field of detection devices, and more particularly, to a method for preparing a biosensing membrane, a biosensing membrane and a monitoring device.

### BACKGROUND

An electrochemical biosensor is a device sensitive to a biological substance and capable of converting its concentration to an electrical signal for detection. It comprises a selective biological substance such as an oxidoreductase or an antibody capable of identifying a target substance, as well as an electrode and an auxiliary device thereof for converting the identified signal into an electrical signal. For instance, when the oxidoreductase is used as the target-identifying substance, the electron exchange between the oxidoreductase and the electrode is an important step performed by a biosensor. Normally, the redox active sites of the oxidoreductase do not exchange electrons with the electrode directly, and thus the electron transfer between the redox active sites and the electrode is a limiting factor for most biosensors. To solve the technical problem relating to the low efficiency of electron transfer, a redox active molecule with excellent electrochemical properties may be introduced into the biosensing membrane to establish an electron transfer channel between the oxidoreductase and the electrode such that a quick electron exchange is achieved.

However, after the introduction of the redox active molecule, how to prepare a stable biosensing membrane using the redox active molecule and the oxidoreductase, how to fix the membrane on the electrode, and how to stably perform a plurality of detections, especially perform detections while ensuring the stability of detection results after being implanted in a living body are technical challenges that need to be solved urgently for those skilled in the art.

### SUMMARY

The purpose of the present disclosure is to provide a method for preparing a biosensing membrane, a biosensing membrane and a monitoring device. The biosensing membrane formed by the aforesaid preparation method is stable and durable, may be used to perform a plurality of detections, and is particularly suitable to serve as a biosensing membrane of a living body monitoring device.

To achieve the above purpose, the present disclosure adopts the following technical solution: a method for preparing a biosensing membrane, comprising the steps of: electrochemically activating and modifying an oxidoreductase, then performing a cross-linking treatment using a chemical cross-linking agent, and then coating on a surface of an electrode, thereby forming a biosensing membrane, wherein the chemical cross-linking agent is glutaraldehyde or polyethylene glycol diglycidyl ether.

In another preferred embodiment, the oxidoreductase is one or more substances selected from glucose oxidase, glucose dehydrogenase, lactate oxidase, lactate dehydrogenase and catalase.

In another preferred embodiment, the oxidoreductase is electrochemically activated and modified using a ruthenium complex having free amino or carboxyl groups or an osmium complex having free amino or carboxyl groups.

In another preferred embodiment, electrochemically activating and modifying the oxidoreductase, comprising the steps of: uniformly mixing the oxidoreductase with the ruthenium complex having free amino or carboxyl groups or the osmium complex having free amino groups in a buffer solution, sequentially adding carbodiimide and N-hydroxysuccinimide, uniformly mixing, reacting at a temperature of 2-6° C. for 12-48 hours, and performing the dialysis using a dialysis bag.

In another preferred embodiment, the oxidoreductase is modified twice using the ruthenium complex having free amino or carboxyl groups or the osmium complex having free amino groups or carboxyl groups, wherein the molecular weight cut off in the preliminary modification is 5000 to 50000, and the molecular weight cut off in the secondary modification is 500 to 50000.

In another preferred embodiment, the osmium complex having free amino or carboxyl groups is Os(bpy)2(3-aminopropyl imidazole)Cl or Os(bpy)2(4-imidazole butyric acid)Cl, and the ruthenium complex having free amino or carboxyl groups is Ru(bpy)2(3-aminopropyl imidazole)Cl or Os(bpy)2(4-imidazole butyric acid)Cl.

In another preferred embodiment, performing a cross-linking treatment using a chemical cross-linking agent, comprising the steps of: uniformly mixing the modified oxidoreductase with the chemical cross-linking agent in a buffer solution, reacting for 0.5-5 hours and coating on the surface of the electrode, thereby forming a biosensing membrane.

In another preferred embodiment, after the biosensing membrane formed by the cross-linking treatment is dried, a polyvinyl pyridine and Nafion mixed alcoholic solution are coated on the surface of the biosensing membrane, thereby forming a biosensing membrane with biocompatibility.

A biosensing membrane prepared by any one of the aforesaid preparation methods.

A monitoring device comprising a sensor, wherein the sensor comprises a biosensing membrane prepared by any one of the aforesaid preparation methods.

The present disclosure provides a method for preparing a biosensing membrane: electrochemically activating and modifying an oxidoreductase, then performing a cross-linking treatment using a chemical cross-linking agent, and then coating on a surface of an electrode, thereby forming a biosensing membrane, wherein the chemical cross-linking agent is glutaraldehyde or polyethylene glycol diglycidyl ether. According to the preparation method of the present disclosure, glutaraldehyde or polyethylene glycol diglycidyl ether is used as the chemical cross-linking agent. The modified oxidoreductase undergoes a cross-linking treatment and is then coated on a surface of an electrode, thereby forming a biosensing membrane on the surface of the electrode. The biosensing membrane formed by means of the cross-linking treatment using glutaraldehyde or polyethylene glycol diglycidyl ether is stable and durable, may be used to perform a plurality of detections, and is particularly suitable to serve as a biosensing membrane of a living body monitoring device.

### BRIEF DESCRIPTION OF THE DRAWINGS

To clearly describe the embodiment of the present disclosure or the technical solution in the prior art, figures needed in the embodiment or prior art are briefly introduced hereinafter. Apparently, the figures in the following description are merely some embodiments recorded in the present disclosure. For those skilled in the art, other figures may be obtained based on the aforesaid figures without paying creative effort.

FIG. 1 is a cyclic voltammogram shows a comparison between the glucose oxidase modified using Os(bpy)2(3-aminopropyl imidazole)Cl and the natural glucose oxidase, wherein (a) is a cyclic voltammogram of the glucose oxidase modified using Os(bpy)2(3-aminopropyl imidazole)Cl and (b) is a cyclic voltammogram of the natural glucose oxidase.

FIG. 2 is a UV-visible absorption spectrum shows a comparison between the glucose oxidase modified using Os(bpy)2(3-aminopropyl imidazole)Cl and the natural glucose oxidase, wherein (a) is a UV-visible absorption spectrum of the glucose oxidase modified using Os(bpy)2(3-aminopropyl imidazole)Cl and (b) is a UV-visible absorption spectrum of the natural glucose oxidase.

FIG. 3 is a cyclic voltammogram shows a comparison between the biosensing membrane containing the modified glucose oxidase of the present disclosure in a pH 7.4 phosphate-buffered saline (PBS) buffer solution and after adding 5.0 mM glucose, wherein (a) is a cyclic voltammogram of the biosensing membrane containing the modified glucose oxidase in a PBS (pH 7.4) buffer solution and (b) is a cyclic voltammogram after adding 5.0 mM glucose.

FIG. 4 is a conceptual diagram showing the relationship between the electrochemical catalytic oxidation current of glucose on the biosensing membrane and the glucose concentration in the embodiment of the present disclosure.

FIG. 5 is a cyclic voltammogram shows a comparison between the biosensing membrane containing the modified lactate oxidase of the present disclosure in a PBS (pH 7.4) buffer solution and after adding 5.0 mM lactate, wherein (1) is a cyclic voltammogram of the biosensing membrane containing the modified lactate oxidase in a PBS (pH 7.4) buffer solution and (2) is a cyclic voltammogram after adding 5.0 mM lactate.

FIG. 6 is a cyclic voltammogram shows a comparison between the biosensing membrane containing the modified glucose dehydrogenase of the present disclosure in a PBS (pH 7.4) buffer solution and after adding 5.0 mM glucose, wherein (1) is a cyclic voltammogram of the biosensing membrane containing the modified glucose dehydrogenase in a PBS (pH 7.4) buffer solution and (2) is a cyclic voltammogram after adding 5.0 mM glucose.

### DETAILED DESCRIPTION

To allow those skilled in the art to better understand the technical solution of the present disclosure, figures are combined hereinafter to clearly and completely describe the technical solution of the present disclosure. Obviously, the described embodiments are merely a part but not all of the embodiments of the present disclosure. Based on the embodiments of the present disclosure, other embodiments obtained by those skilled in the art without paying creative effort shall fall into the scope of the present disclosure.

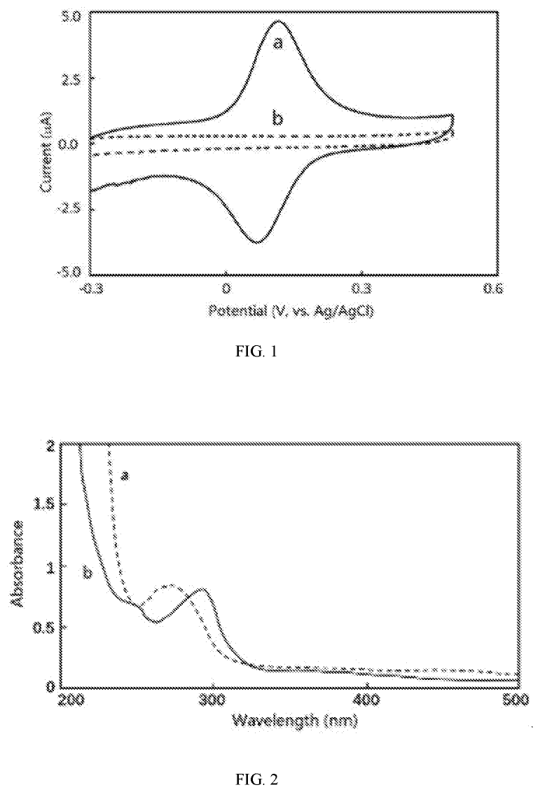

As shown in FIGS. 1-6, the present disclosure provides a method for preparing a biosensing membrane: modifying an oxidoreductase, then performing a cross-linking treatment using a chemical cross-linking agent, and then coating on a surface of an electrode, thereby forming a biosensing membrane, wherein the chemical cross-linking agent is glutaraldehyde or polyethylene glycol diglycidyl ether.

The present disclosure provides a method for preparing a biosensing membrane: electrochemically activating and modifying an oxidoreductase, then performing a cross-linking treatment using a chemical cross-linking agent, and then coating on a surface of an electrode, thereby forming a biosensing membrane, wherein the chemical cross-linking agent is glutaraldehyde or polyethylene glycol diglycidyl ether. According to the preparation method of the present disclosure, glutaraldehyde or polyethylene glycol diglycidyl ether is used as the chemical cross-linking agent. The modified oxidoreductase undergoes a cross-linking treatment and is then coated on a surface of an electrode, thereby forming a biosensing membrane on the surface of the electrode. The biosensing membrane formed by means of the cross-linking treatment using glutaraldehyde or polyethylene glycol diglycidyl ether is stable and durable, may be used to perform a plurality of detections, and is particularly suitable to serve as a biosensing membrane of a living body monitoring device.

Preferably, the oxidoreductase is one or more substances selected from glucose oxidase, glucose dehydrogenase, lactate oxidase, lactate dehydrogenase and catalase.

In the present disclosure, more specifically, the oxidoreductase is one or more substances selected from glucose oxidase, glucose dehydrogenase, lactate oxidase, lactate dehydrogenase and catalase. After being electrochemically activated and modified, and then treated using a chemical cross-linking agent, a stable biosensing membrane is formed on the surface of the electrode for detecting a target substance.

Preferably, the oxidoreductase is electrochemically activated and modified using a ruthenium complex having free amino or carboxyl groups or an osmium complex having free amino or carboxyl groups.

Preferably, modifying the oxidoreductase comprising the steps of : uniformly mixing the oxidoreductase with the ruthenium complex having free amino or carboxyl groups or the osmium complex having free amino or carboxyl groups in a buffer solution, sequentially adding carbodiimide and N-hydroxysuccinimide, uniformly mixing, reacting at a temperature of 2-6° C. for 12-48 hours, and performing the dialysis using a dialysis bag.

Preferably, the oxidoreductase is modified for twice using the ruthenium complex having free amino or carboxyl groups or the osmium complex having free amino or carboxyl groups, wherein the molecular weight cut off in the preliminary modification is 5000 to 50000, and the molecular weight cut off in the secondary modification is 500 to 50000.

Preferably, the osmium complex having free amino or carboxyl groups is Os(bpy)2(3-aminopropyl imidazole)Cl or Os(bpy)2(4-imidazole butyric acid)Cl, and the ruthenium complex having free amino or carboxyl groups is Ru(bpy)2(3-aminopropyl imidazole)Cl or Os(bpy)2(4-imidazole butyric acid)Cl.

In the present disclosure, preferably, the oxidoreductase is modified using the ruthenium complex having free amino or carboxyl groups or the osmium complex having free amino or carboxyl groups, and more preferably, the oxidoreductase is modified using Os(bpy)2(3-aminopropyl imidazole)Cl or Ru(bpy)2(3-aminopropyl imidazole)Cl, wherein bpy refers to 2,2-bipyridine.

Electrochemically activating and modifying the oxidoreductase, comprising the steps of: uniformly mixing the oxidoreductase with the ruthenium complex having free amino groups or the osmium complex having free amino groups in a buffer solution, sequentially adding carbodiimide and N-hydroxysuccinimide, uniformly mixing, reacting at a temperature of 2-6° C. for 12-48 hours, and performing the dialysis using a dialysis bag. More preferably, the oxidoreductase is modified for twice using the ruthenium complex having free carboxyl groups or the osmium complex having free carboxyl groups, wherein the molecular weight cut off in the preliminary modification is 5000 to 50000, and the molecular weight cut off in the secondary modification is 500 to 50000.

More specifically, electrochemically activating and modifying the oxidoreductase for twice using the ruthenium complex having free amino or carboxyl groups or the osmium complex having free amino or carboxyl groups, comprising the steps of: uniformly mixing the oxidoreductase (e.g., glucose oxidase) with the ruthenium complex having free amino groups or the osmium complex having free amino groups in a PBS buffer solution, sequentially adding carbodiimide and N-hydroxysuccinimide, uniformly mixing, reacting at a temperature of 2-6° C. for 12-48 hours, and performing the dialysis using a dialysis bag, wherein the molecular weight cut off is 5000 to 50000; subsequently, separating and purifying the preliminarily modified oxidoreductase, adding the ruthenium complex having free carboxyl groups or the osmium complex having free carboxyl groups into the purified oxidoreductase solution, sequentially adding carbodiimide and N-hydroxysuccinimide, uniformly mixing, and reacting at a temperature of 2-6° C. for 12-48 hours; after the reaction is completed, re-performing the dialysis using a dialysis bag, wherein the molecular weight cut off is 500 to 50000; finally, separating and purifying the secondarily modified oxidoreductase, wherein the concentration of the ruthenium complex having free amino or carboxyl groups or the osmium complex having free amino or carboxyl groups is preferably 0.1-50 mg/ml and more preferably 1-20 mg/ml, the concentration of the carbodiimide is preferably 0.1-50 mmol/L and more preferably 0.1-25 mmol/L, and the concentration of the N-hydroxysuccinimide is preferably 0.01-5 mmol/L. The spectrophotometric analysis proves that the oxidoreductase has been successfully modified.

Preferably, performing a cross-linking treatment using a chemical cross-linking agent, comprising the steps of: uniformly mixing the modified oxidoreductase with the chemical cross-linking agent in a buffer solution, reacting for 0.5-5 hours and coating on the surface of the electrode, thereby forming a biosensing membrane.

Preferably, after the biosensing membrane formed by the cross-linking treatment is dried, a polyvinyl pyridine and a Nafion mixed alcoholic solution are coated on the surface of the biosensing membrane, thereby forming a biosensing membrane with biocompatibility.

In the present disclosure, more specifically, performing a cross-linking treatment using a chemical cross-linking agent, comprising the steps of: uniformly mixing the modified oxidoreductase with the chemical cross-linking agent in a buffer solution, reacting for 0.5-5 hours and coating on the surface of the electrode, thereby forming a biosensing membrane; preferably, after the biosensing membrane formed by the cross-linking treatment is dried, coating a polyvinyl pyridine and a Nafion mixed alcoholic solution on the surface of the biosensing membrane, thereby forming a biosensing membrane with biocompatibility.

More specifically, making the modified oxidoreductase react with a chemical cross-linking agent, comprising the steps of: fully mixing the modified oxidoreductase with a glutaraldehyde solution or a polyethylene glycol diglycidyl ether solution in a PBS buffer solution, reacting for 0.5-5 hours, preferably 0.5-3 hours, and coating the chemically crosslinked oxidoreductase on the surface of the electrode through adopting a drop-casting method or a dip-coating method, thereby forming a biosensing membrane; after the biosensing membrane on the electrode is completely dried, coating a polyvinyl pyridine and a nafion mixed alcoholic solution on the surface of the biosensing membrane through adopting a drop-casting method or a dip-coating method, thereby forming a biosensing membrane with biocompatibility. Preferably, the concentration of the chemical cross-linking agent (glutaraldehyde solution or polyethylene glycol diglycidyl ether solution) is 0.1-5%, the concentration of the modified oxidoreductase is preferably 5-150 mg/ml, the concentration of the polyvinyl pyridine solution is preferably 20-300 mg/ml, and the concentration of the nafion mixed alcoholic solution is preferably 0.1-5%. Preferably, a mixed ethanol solution containing polyvinyl pyridine and Nafion is adopted, namely, polyvinyl pyridine and Nafion being dissolved and mixed in alcohol to obtain a mixed ethanol solution containing polyvinyl pyridine and Nafion.

After performing the cross-linking treatment, the oxidoreductase maintains its direct electrochemical action. Experiments show that the modified glucose oxidase maintains its performance of catalytic oxidation for glucose in the biosensing membrane, and the catalytic oxidation efficiency of the modified glucose oxidase for glucose through direct electrochemistry is 140 times higher than that of the natural glucose oxidase for glucose through oxygen.

A biosensing membrane prepared by any one of the aforesaid preparation methods.

A monitoring device comprising a sensor, wherein the sensor comprises a biosensing membrane prepared by any one of the aforesaid preparation methods.

The present disclosure provides a biosensing membrane prepared by any one of the aforesaid preparation methods and a monitoring device comprising a sensor, wherein the sensor comprises a biosensing membrane prepared by any one of the aforesaid preparation methods.

Preferably, the monitoring device is an implantable continuous monitoring device, which may stably operate after being implanted in a life body (such as a human body), and may perform a plurality of detections for a target substance for a long time. According to the implantable continuous monitoring device, a sensor fixed with a biosensing membrane is implanted under the skin, and a receiver or a mobile device receives the data from the sensor in real time, thereby realizing a long-term continuous monitoring of the concentration of the target substance. For instance, an implantable blood glucose monitoring device may be developed and used for monitoring the blood glucose and supplementing insulin in time using an insulin pump, thereby regulating the blood glucose contained in the human body.

### Embodiment 1

In this embodiment, modifying the oxidoreductase, comprising the steps of: sufficiently mixing the glucose oxidase with 1-20 mg/ml Os(bpy)2(3-aminopropyl imidazole)Cl in a PBS buffer solution, sequentially adding 0.1-25 mmol/L carbodiimide and 0.01-5 mmol/L N-hydroxysuccinimide, sufficiently mixing, reacting at a temperature of 4° C. for 24 hours, and performing the dialysis using a dialysis bag, wherein the molecular weight cut off is 5000 to 50000; subsequently, separating and purifying the preliminarily modified oxidoreductase, adding 1-20 mg/ml Os(bpy)2(4-imidazole butyric acid)Cl into the purified oxidoreductase solution, sequentially adding 0.1-25 mmol/L carbodiimide and 0.01-5 mmol/L N-hydroxysuccinimide, sufficiently mixing, and reacting at a temperature of 4° C. for 24 hours; after the reaction is completed, re-performing the dialysis using a dialysis bag, wherein the molecular weight cut off is 500 to 50000; finally, separating and purifying the secondarily modified oxidoreductase. After the glucose oxidase is modified, its catalytic active center may directly quickly exchange electrons with the electrode (shown in FIG. 1). The spectrophotometric analysis proves that the oxidoreductase has been successfully modified (shown in FIG. 2).

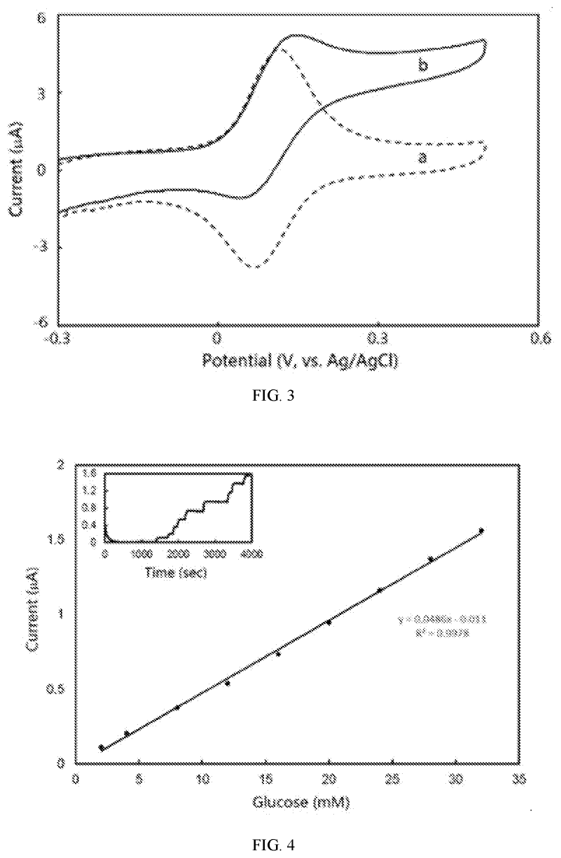

Making the modified oxidoreductase react with a chemical cross-linking agent, comprising the steps of: fully mixing the modified 5-150 mg/ml oxidoreductase with a 0.1-5% glutaraldehyde solution in a PBS buffer solution, reacting for 0.5-3 hours, and coating the chemically crosslinked oxidoreductase on the surface of the electrode through adopting a drop-casting method or a dip-coating method, thereby forming a biosensing membrane; after the biosensing membrane on the electrode is completely dried, coating a mixed ethanol solution containing 20-300 mg/ml polyvinyl pyridine and 0.1-5% Nafion on the surface of the biosensing membrane through adopting a drop-casting method or a dip-coating method, thereby forming a biosensing membrane with biocompatibility. After performing the cross-linking treatment using a glutaraldehyde solution, the oxidoreductase maintains its direct electrochemical action, and the prepared biosensing membrane shows an ideal electrochemical performance (shown in FIG. 3(a)). After adding 5.0 mM glucose into the PBS buffer solution, the cyclic voltammogram of the biosensing membrane clearly shows a typical electrochemical catalytic process (shown in FIG. 3(b)). Experiments show that the modified glucose oxidase maintains its performance of catalytic oxidation for glucose in the biosensing membrane, and the catalytic oxidation efficiency of the modified glucose oxidase for glucose through direct electrochemistry is 140 times higher than that of the natural glucose oxidase for glucose through oxygen.

Based on the aforesaid, the biosensing membrane is adopted in an implantable glucose continuous monitoring system. The preliminary experimental result shows that the operating curve is in an ideal linear state between 2.0-32 mM (shown in FIG. 4). It is an implantable glucose continuous monitoring system having the widest linear range.

In addition to glucose oxidase, other oxidoreductases such as lactate oxidase and glucose dehydrogenase may also be successfully modified and chemically crosslinked with glutaraldehyde.

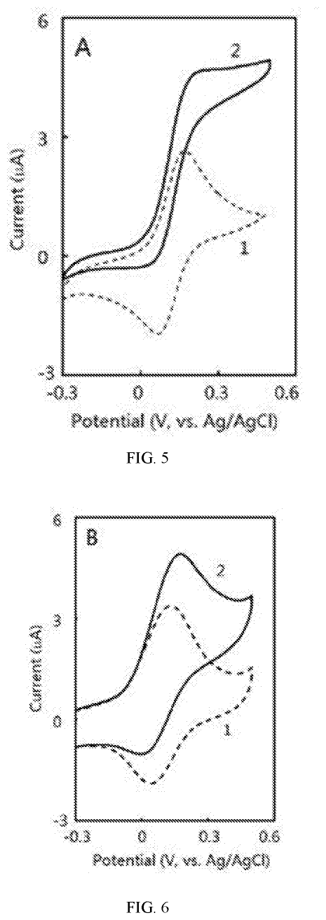

Experiments show that the biosensing membranes containing modified lactate oxidase and glucose dehydrogenase show good electrochemical performance on the electrode (shown in FIGS. 5 and 6). After adding 5.0 mM lactate or glucose into the PBS buffer solution, the cyclic voltammogram of the biosensing membranes clearly show a typical electrochemical catalytic process (shown in FIGS. 5 and 6). These results indicate that they maintain their catalytic oxidation performance through direct electrochemistry in the biosensing membranes.

The aforesaid description of the disclosed embodiment enables those skilled in the art to realize or use the present disclosure. Various modifications to these embodiments are apparent to those skilled in the art, and the general principles defined in the present disclosure may be implemented in other embodiments without departing from the spirit or scope of the present disclosure. Therefore, the present disclosure shall not be limited to the described embodiments but conform to the widest range consistent with the principles and novel features disclosed in the present disclosure.

What is claimed is:

1. A method for preparing a biosensing membrane, comprising the steps of: electrochemically activating and modifying an oxidoreductase, then performing a cross-linking treatment using a chemical cross-linking agent, and then coating on a surface of an electrode, thereby forming a biosensing membrane, wherein the chemical cross-linking agent is glutaraldehyde or polyethylene glycol diglycidyl ether.

2. The preparation method of claim 1, wherein the oxidoreductase is one or more substances selected from glucose oxidase, glucose dehydrogenase, lactate oxidase, lactate dehydrogenase and catalase.

3. The preparation method of claim 2, wherein the oxidoreductase is electrochemically activated and modified using a ruthenium complex having free amino or carboxyl groups or an osmium complex having free amino or carboxyl groups.

4. The preparation method of claim 3, wherein electrochemically activating and modifying the oxidoreductase, comprising the steps of: uniformly mixing the oxidoreductase with the ruthenium complex having free amino or carboxyl groups or the osmium complex having free amino groups in a buffer solution, sequentially adding carbodiimide and N-hydroxysuccinimide, uniformly mixing, reacting at a temperature of 2-6° C. for 12-48 hours, and performing the dialysis using a dialysis bag.

5. The preparation method of claim 4, wherein the oxidoreductase is modified for twice using the ruthenium complex having free amino or carboxyl groups or the osmium complex having free amino groups, wherein the molecular weight cut off in the preliminary modification is 5000 to 50000, and the molecular weight cut off in the secondary modification is 500 to 50000.

6. The preparation method of claim 5, wherein the osmium complex having free amino or carboxyl groups is Os(bpy)2(3-aminopropyl imidazole)Cl or Os(bpy)2(4-imidazole butyric acid)Cl, and the ruthenium complex having free amino or carboxyl groups is Ru(bpy)2(3-aminopropyl imidazole)Cl or Os(bpy)2(4-imidazole butyric acid)Cl.

7. The preparation method of claims 1, wherein performing a cross-linking treatment using a chemical cross-linking agent, comprising the steps of: uniformly mixing the modified oxidoreductase with the chemical cross-linking agent in a buffer solution, reacting for 0.5-5 hours and coating on the surface of the electrode, thereby forming a biosensing membrane.

8. The preparation method of claim 7, wherein after the biosensing membrane formed by the cross-linking treatment is dried, a polyvinyl pyridine and a Nafion mixed alcoholic solution are coated on the surface of the biosensing membrane, thereby forming a biosensing membrane with biocompatibility.

9. A biosensing membrane prepared by the preparation method of claims 1.

10. A monitoring device, comprising:
a sensor, wherein the sensor comprises a biosensing membrane prepared by the preparation method of claims 1.
